(19) United States
(12) Patent Application Publication
(10) Pub. No.: US 2012/0271400 A1
(43) Pub. Date: Oct. 25, 2012

(54) INTRODUCER FOR A VASCULAR REPAIR PROSTHESIS

(76) Inventors: Drew P. Lyons, Ellettsville, IN (US); Elizabeth M. Brown, Bloomington, IN (US)

(21) Appl. No.: 13/091,704

(22) Filed: Apr. 21, 2011

(51) Int. Cl. A61F 2/84

(52) U.S. Cl. 623/1.12

(57) ABSTRACT

An introducer system and method for introducing a prosthesis for repair of two vessel portions of a transected body vessel is provided. The system can include one or more retaining members fitted over the ends of the prosthesis to retain the ends radially compressed. The retaining member can include an elongated body for insertion into the vessel opening and a gripping member, which both can be rigid. A splittable region can be formed in the elongated body and the gripping member. Application of a force to the gripping member can separate or fracture the gripping member and the elongated body along the splittable region to permit radial expansion of the prosthesis for engagement with the vessel. A sleeve may at least partially surround an intermediate portion of the prosthesis. The sleeve can be configured to retain the intermediate portion of the prosthesis in the radially compressed configuration.

### BACKGROUND

The present disclosure relates generally to medical devices for emergency repair of body vessels. More particularly, it relates to an introducer used for insertion of a medical device for repairing damaged body vessels.

Trauma physicians frequently encounter patients having traumatic injury to a body vessel, such as lacerated vessels or even transected vessels, resulting from gunshots, knife wounds, motor vehicle accidents, explosions, etc. Significant damage to a body vessel may expose a patient to deleterious conditions such as the loss of a limb, loss of function of a limb, increased risk of stroke, impairment of neurological functions, and compartment syndrome, among others. Particularly severe cases of vascular injury and blood loss may even result in death. In such severe situations, the immediate goal is to obtain hemostasis while maintaining perfusion of adequate blood flow to critical organs, such as the brain, liver, kidneys, and heart.

Examples of treatment that are commonly performed by trauma physicians to treat body vessel injuries include the clamping of the vessel with a hemostat, the use of a balloon tamponade, the ligation of the damaged vessel at or near the site of injury, or the insertion of one or more temporary shunts. However, conventional surgical repair is generally difficult with such actively bleeding, moribund patients. In many instances, there is simply not enough time to repair the body vessel adequately by re-approximating and suturing the body vessel. In many situations, the trauma physician will simply insert a temporary shunt (such as a Pruitt-Inahara Shunt) into the vessel. However, use of temporary shunts has been linked to the formation of clots. This may require returning the patient to the operating room for treatment and removal of the clots, often within about 36 to 48 hours of the original repair. Since shunts are generally placed as a temporary measure to restore blood flow and stop excessive blood loss, the shunt is typically removed when the patient has stabilized (generally a few days later) by a specialized vascular surgeon. After removal, the vascular surgeon will replace the shunt with a vascular graft, such as a fabric graft that is sewn into place. With respect to ligation, ligation of the damaged blood vessel may result in muscle necrosis, loss of muscle function, or a potential limb loss or death.

Due to the nature of the body vessel injury that may be encountered, the insertion of shunts or ligation of a blood vessel, for example, often requires that such treatments be rapidly performed at great speed, and with a high degree of physician skill. Such treatments may occupy an undue amount of time and attention of the trauma physician at a time when other pressing issues regarding the patient's treatment require immediate attention. In addition, the level of particularized skill required to address a vascular trauma may exceed that possessed by the typical trauma physician. Particularly, traumatic episodes to the vessel may require the skills of a physician specially trained to address the particular vascular trauma, and to stabilize the patient in the best manner possible under the circumstances of the case.

Some open surgical techniques utilize sutures to affix damaged tissue portions surrounding fittings that have been deployed with the vessel, which requires the trauma physician to take time to tie the sutures properly. Although in modern medicine sutures can be tied in relatively rapid fashion, any step in a repair process that occupies physician time in an emergency situation is potentially problematic. In addition, the use of sutures to affix the vessel to the fitting compresses the tissue of the vessel against the fitting. Compression of tissue may increase the risk of necrosis of the portion of the vessel tissue on the side of the suture remote from the blood supply. When present, necrosis of this portion of the vessel tissue may result in the tissue separating at the point of the sutures. In this event, the connection between the vessel and the fitting may eventually become weakened and subject to failure. If the connection fails, the device may disengage from the vessel. Therefore, efforts continue to develop techniques that reduce the physician time required for such techniques, so that this time can be spent on other potentially life-saving measures, and the blood flow is more quickly restored and damage caused by lack of blood flow is minimized.

Trauma physicians generally find it difficult to manipulate a prosthesis for insertion into a body vessel that has been traumatically injured. For example, one difficulty arises from the trauma physician trying to limit the size of the opening created for gaining access to the injured vessel so that such opening requiring healing is as small as possible. Another difficulty is that the injured vessel can be anywhere in the body, having different surrounding environments of bone structure, muscle tissue, blood vessels, and the like, which makes such obstructions difficult to predict in every situation and leaves the trauma physician working with an even further limited access opening. Another potential consideration is the amount of body vessel removed during a transection. The goal would be to remove a portion of the body vessel as small as possible. Yet, a small portion removed from the vessel leaves such a small space between the two vessel portions, thereby making it difficult to introduce the prosthesis between the two vessel portions.

Thus, what is needed is an introducer configured for delivering a prosthesis for use in repair of an injured body vessel, such as an artery or a vein, (and in particular a transected vessel) during emergency surgery. It would be desirable if such deployment device is easy for a trauma physician to use, and can rapidly introduce a prosthesis into two vessel portions of a transected vessel, thereby providing a conduit for blood within the injured body vessel.

### BRIEF SUMMARY

In one embodiment, an introducer system is described herein for introducing a prosthesis for repair of two vessel portions of a transected body vessel. The system can include a prosthesis and one or more retaining members. The prosthesis can have a first end and a second end, and can be movable between a radially compressed configuration and a radially expanded configuration. The retaining member can be fitted over at least one of the first and second ends of the prosthesis to retain a corresponding length of the prosthesis in the radially compressed configuration. The retaining member can include an elongated body for insertion into a vessel portion of a transected body vessel and a gripping member. The elongated body can have a tubular chamber formed therein to receive the respective end of the prosthesis. The gripping member can extend outwardly from the elongated body. A splittable region can be formed in the elongated body and the gripping member. The retaining member may have a rigidity suitable to maintain a relative orientation between the gripping member and the elongated body during normal use. Application of a force to the gripping member can separate the gripping member and the elongated body along the splittable region to permit movement of the corresponding length of the prosthesis to the radially expanded configuration for engagement with the vessel portion. The splittable region can include first and second predetermined splittable regions. A removable sleeve may be provided to at least partially surround an intermediate portion of the prosthesis. The sleeve can be configured to selectively retain the intermediate portion of the prosthesis in the radially compressed configuration.

In another embodiment, a method of connecting two vessel portions of a transected body vessel is also provided. A first end of a prosthesis in a radially compressed configuration, retained by a tubular elongated body of a retaining member, can be introduced into an end opening of one of the two vessel portions. The retaining member can have a gripping member extending outward from the elongated body, and a splittable region formed in the elongated body and the gripping member. A force can be applied to the gripping member to separate the retaining member along the splittable region into two or more removable portions. The first end of the prosthesis can be permitted to move to a radially expanded configuration for engagement with the vessel portion. A second retaining member can be fitted over the second end of the prosthesis for insertion into the other of the two vessel portions to connect the two vessel portions to one another. The retaining member may be capable of sliding along the prosthesis before splitting. A sleeve can be provided along a portion of the prosthesis to facilitate sliding of the retaining member.

### BRIEF DESCRIPTION OF THE DRAWINGS

FIG. 1 is a side elevational view of an example of an introducer system including a retaining member coupled to ends of a prosthesis.

FIG. 2 is a perspective view of an example of a retaining member.

FIG. 3 is a transverse sectional view of the retaining member of FIG. 2.

FIG. 4 is an end view of the retaining member of FIG. 2, depicting a surface of a gripping member.

FIG. 5 is a transverse sectional view of an elongated body of the retaining member of FIG. 2.

FIG. 6 is a side elevational view of the retaining member of FIG. 2 being split along a predetermined splittable region.

FIG. 7 is a partial sectional view of an opening of a gripping member of the retaining member of FIG. 2.

FIG. 8 is a perspective view of another example of a retaining member.

FIG. 9 is an end view of the retaining member of FIG. 8, depicting a surface of a gripping member.

FIGS. 10A-10H are side elevational views depicting various steps of a method of repairing two vessel portions of a transected body vessel.

FIGS. 11A-11B are side elevational views depicting various alternative steps of a method of repairing two vessel portions of a transected body vessel.

FIG. 11C is a transverse sectional view of an elongated body of a retaining member taken along lines 11C-11C in FIG. 11A.

FIG. 12A is a side elevational view of another example of an introducer system including a retaining member coupled to ends of a prosthesis and a sleeve.

FIG. 12B is a side elevational view of a retaining member of the introducer system of FIG. 12A sliding over the prosthesis and the sleeve.

FIG. 12C is a transverse sectional view of an intermediate portion of the prosthesis and the sleeve taken along lines 12C-12C in FIG. 12A.

### DETAILED DESCRIPTION OF THE DRAWINGS AND THE PRESENTLY PREFERRED EMBODIMENTS

For the purposes of promoting an understanding of the principles of the present disclosure, reference will now be made to the embodiments illustrated in the drawings, and specific language will be used to describe the same.

In the following discussion, the terms “proximal” and “distal” will be used to describe the opposing axial ends of the inventive sheath, as well as the axial ends of various component features. The term “proximal” is used in its conventional sense to refer to the end of the apparatus (or component thereof) that is closest to the operator during use of the apparatus. The term “distal” is used in its conventional sense to refer to the end of the apparatus (or component thereof) that is initially inserted into the patient, or that is closest to the patient during use.

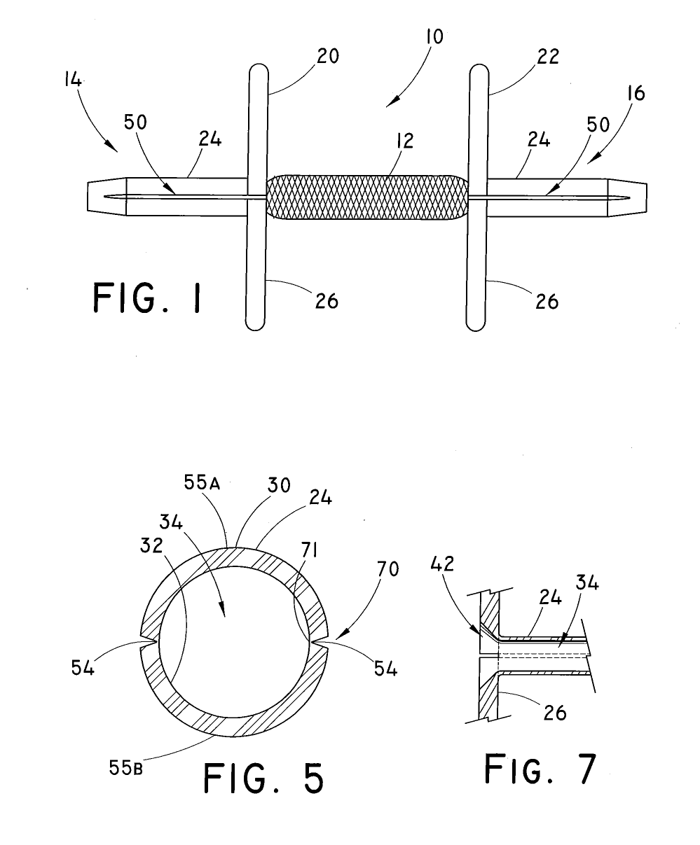

FIG. 1 shows an exemplary introducer system 10 including a prosthesis 12 having a first end 14 and a second end 16, and a first retaining member 20 and a second retaining member 22 disposed at the respective first and second ends 14, 16 of the prosthesis 12. The introducer system 10 can be useful for repair of a body vessel, such as a blood vessel, during an emergency open surgical procedure or intraoperative repair. This system can be particularly useful for introducing a prosthesis for repair of a lacerated vessel during emergency surgery, and particularly, for obtaining hemostasis while maintaining blood perfusion for a transected vessel. In particular, during an open surgical procedure, a trauma pathway is formed to gain external access to the damaged vessel. The trauma pathway is typically small and substantially transverse to the damaged vessel. To accommodate such surroundings, the retaining member can be configured to provide increased manipulation of the prosthesis, such as pushability, during insertion into a vessel portion after transection, and rapid removal from the body, such as splittability and/or slidability from the prosthesis, to allow expansion thereof. Other applications for the system will become readily apparent to one skilled in the art from the detailed description.

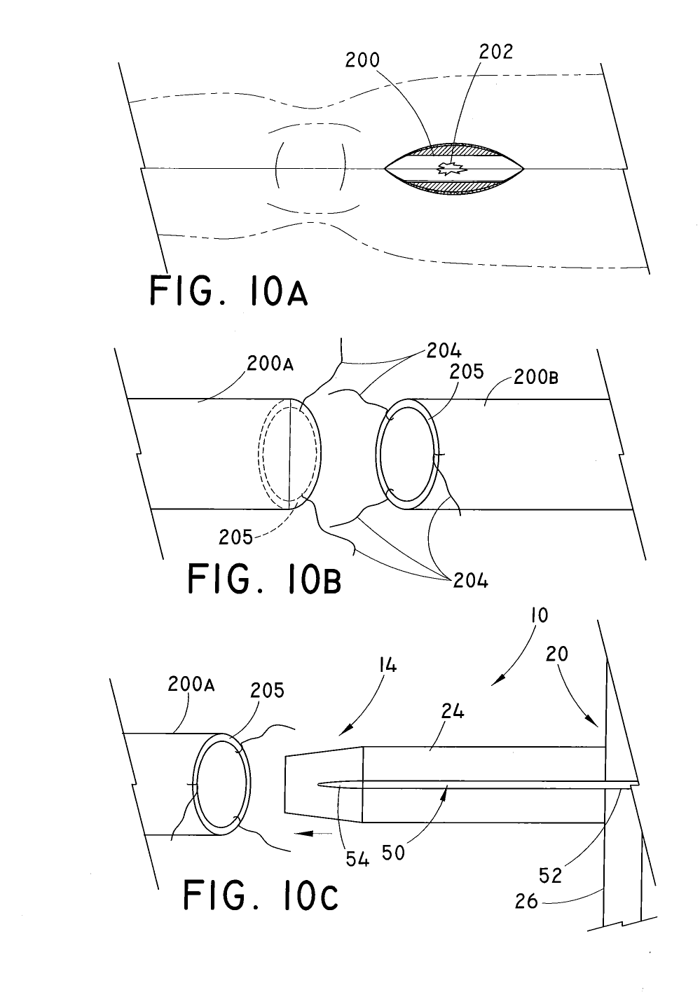

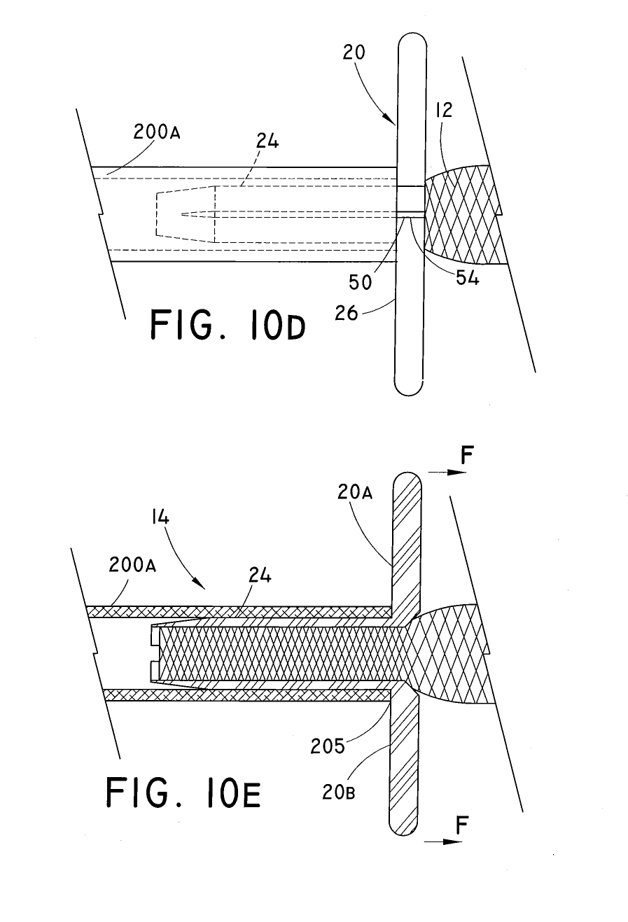

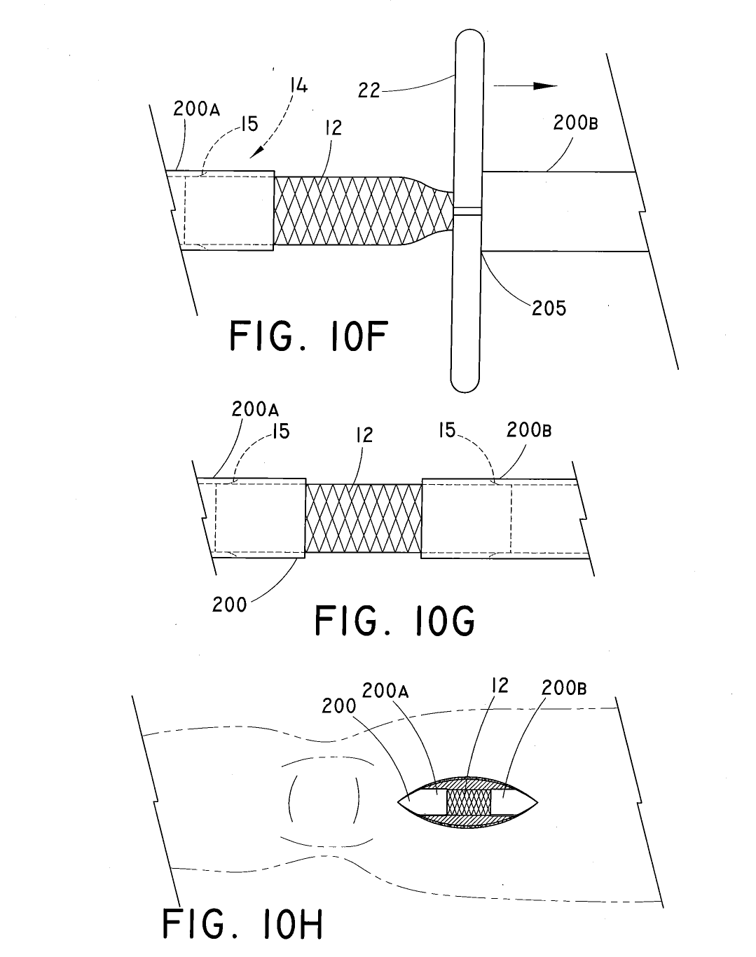

A concise description of the prosthesis 12 will now be provided. The prosthesis can include a generally tubular graft body, a tubular support structure, and/or one or more anchoring members 15 (see FIG. 10F) together defining a fluid passageway. The prosthesis is movable between a radially compressed, delivery configuration and a radially expanded, deployed configuration. The prosthesis can be balloon expandable; however, it is preferred that the prosthesis is self-expandable. The anchoring members and/or support structure can be attached to the graft body by sutures sewn therein, wire, staples, clips, bonding agents, or other methods that may be used to achieve a secure attachment to the graft body. The prosthesis has a size and shape suitable for at least partial placement within a body vessel, such as an artery or vein, and most particularly, for placement at the site of a vascular trauma. The prosthesis may be easily manipulated during delivery to a transected artery or vein during emergency surgery, and particularly, to obtain hemostasis while maintaining blood perfusion. The anchoring member and/or support structure can be any stent pattern known to one skilled in the art. Examples of stent patterns includes the Z-STENT® and ZILVER® stent, each available from COOK MEDICAL (Bloomington, Ind.). The anchoring member and/or support structure can be formed of a biocompatible metal, such as stainless steel (e.g., 316L SS), titanium, tantalum, nitinol or other shape memory materials, or a high-strength polymer. The anchoring member is configured to purchase the vessel wall in order to reduce the risk of migration of the prosthesis. Preferably, anchoring devices can be included on at least the anchoring members to provide vessel fixation, while avoiding adverse conditions associated with disturbing the vasa vasorum and/or pressure induced necrosis of the medium muscular arteries of the type that may result from tying ligatures circumferentially around a connector or a vascular conduit. The anchoring devices can include various shaped member structures, including barbs, fibers, bristles, or outer protruding and penetrable media.

The graft body can be formed from conventional materials well known in the medical arts. The graft body may comprise an expanded polytetrafluoroethylene (ePTFE), polytetrafluoroethylene, silicone, polyurethane, polyamide (nylon), as well as other flexible biocompatible materials. The graft body can also be formed from known fabric graft materials such as woven polyester (e.g. DACRON®), polyetherurethanes such as THORALON® from Thoratec Corporation, Pleasanton, Calif., and polyethylene such as an ultra-high molecular weight polyethylene (UHMwPE), commercially available as DYNEEMA®. The graft body may also include a bioremodelable material, such as reconstituted or naturally-derived collagenous materials, extracellular matrix material (ECM), submucosa, renal capsule membrane, dermal collagen, dura mater, pericardium, fascia lata, serosa, peritoneum or basement membrane layers, intestinal submucosa, including small intestinal submucosa (SIS), stomach submucosa, urinary bladder submucosa, and uterine submucosa. One non-limiting example of a suitable remodelable material is the SURGISIS® BIODESIGN™, commercially available from COOK MEDICAL (Bloomington, Ind.). Another suitable remodelable material is the graft prosthesis material described in U.S. Pat. No. 6,206,931 to Cook et al., incorporated herein by reference.

Portions of the prosthesis can also include a coating of one or more therapeutic agents along a portion of the stent structure and/or the graft body. Therapeutic agents for use as biocompatible coatings are well known in the art. Non-limiting examples of suitable bio-active agents that may be applied to the vascular conduit include thrombo-resistant agents, antibiotic agents, anti-tumor agents, antiviral agents, anti-angiogenic agents, angiogenic agents, anti-mitotic agents, anti-inflammatory agents, angiostatin agents, endostatin agents, cell cycle regulating agents, genetic agents, including hormones such as estrogen, their homologs, derivatives, fragments, pharmaceutical salts and combinations thereof. Those skilled in the art will appreciate that other bioactive agents may be applied for a particular use. The bioactive agent can be incorporated into, or otherwise applied to, portions of the vascular conduit by any suitable method that permits adequate retention of the agent material and the effectiveness thereof for its intended purpose. Although the device has been described in connection with its primary intended use for repair of vascular trauma, those skilled in the art will appreciate that the device may also be used to repair other traumatic conditions. Non-limiting examples of such conditions include aneurysms, such as abdominal aorta aneurysms, and surgery for tumor removal.

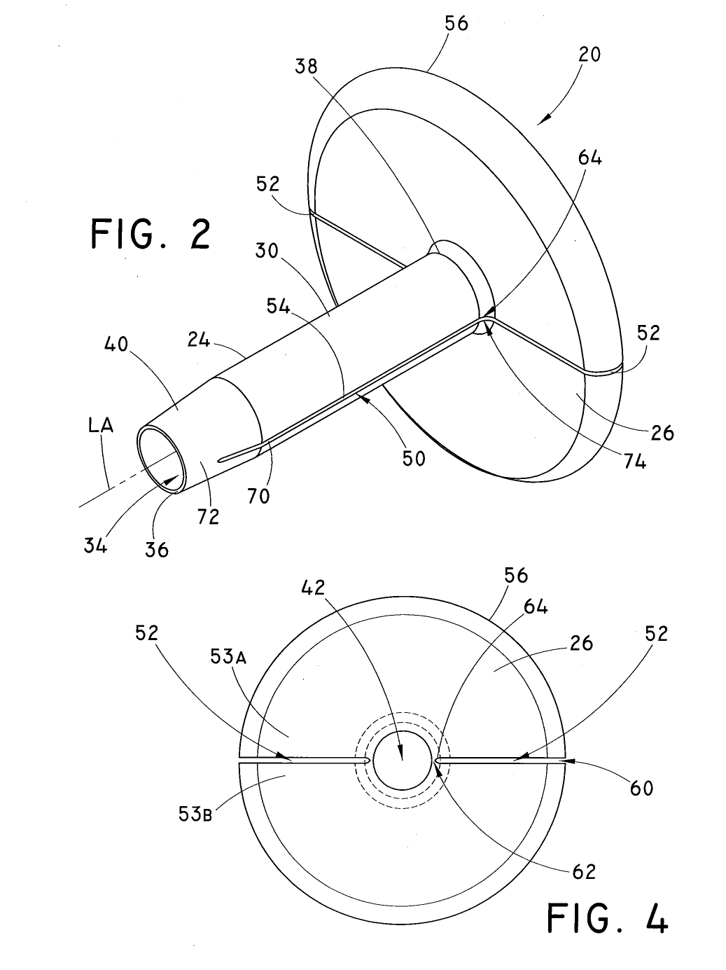

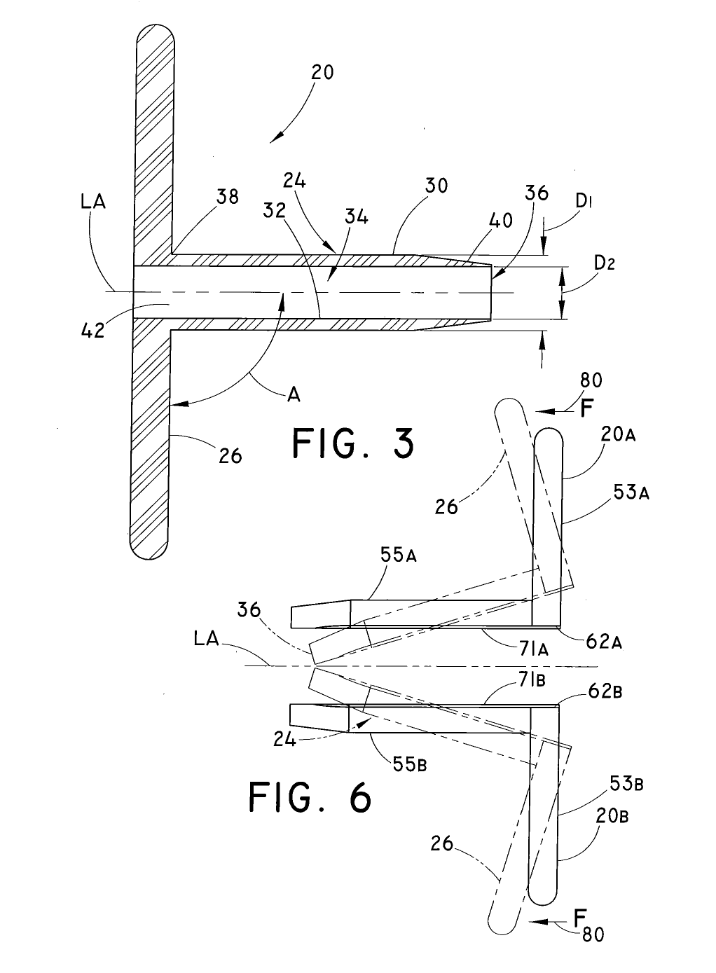

With reference to FIG. 2, discussion will now focus on the first retaining member 20, which is representative also of the second retaining member 22. The retaining member 20 includes an elongated body 24 and a gripping member 26 extending outwardly from the elongated body 24. According to FIGS. 2 and 3, the elongated body 24 includes an outer wall surface 30 and an inner wall surface 32. The inner surface wall 32 can define a tubular inner chamber 34 about a longitudinal axis LA, which is generally in alignment with the axis of the prosthesis. The elongated body 24 can have an outer diameter D1 that is sized to fit directly within a body vessel. The inner chamber 34 of the elongated body 24 can have an inner diameter D2 that is sized to receive a portion of the prosthesis 12 that is in a radially compressed configuration. The respective portion of the prosthesis 12 can be crimped or otherwise radially compressed as appreciated by those skilled in the art to a profile sized to fit within the inner chamber 34. The elongated body 24 can extend between a first end 36 for insertion into the vessel portion end opening and a second end 38. The outer wall surface 30 of the elongated body 24 may have a uniform diameter D1 entirely between the first and second ends 36, 38 of the body. In one example, a tapered segment 40 of the outer surface wall proximate the first end 36 of the body may be tapered from the diameter D1 to approximately the diameter D2 at the tip to allow for easier insertion into the body vessel end opening. It is further contemplated that the outer diameter D1 and/or the inner diameter D2 can vary along the length of the respective wall surfaces in order to form tapered walls and/or tapered outer and/or inner surfaces.

According to FIGS. 2-4, the gripping member 26 is generally an enlarged member having a greater cross-sectional area relative to the cross-sectional area of the elongated body 24 to permit the operator to handle and manipulate the retaining member 20. The gripping member 26 can extend outward from the elongated body 24 at any angle such as obliquely relative to the longitudinal axis LA toward either the first end or the second end. In one example, the gripping member 26 can extend substantially perpendicular to the longitudinal axis LA. The gripping member 26 can have an opening 42 formed therein which can be an extension of the inner chamber 34 of the elongated body 24. The opening 42 permits a portion of the prosthesis to extend longitudinally therethrough and externally beyond the gripping member. In FIG. 7, the opening 42 may be at least partially tapered in a manner so that the diameter of the end closer in proximity to the inner chamber 34 is approximately the same diameter as the inner chamber, and the opposite end farther in proximity is greater than the diameter of the closer end.

The retaining member 20 can be splittable, preferably longitudinally, along a relatively predictable path. The retaining member is usually, but not necessarily, separated into two or more portions, thereby forming a fissure along the length of the elongated body and the gripping member that permits removal of the retaining member from around the prosthesis situated in the inner chamber. In FIG. 2, a predetermined splittable region 50 may be formed in the retaining member 20 through which the separation progresses.

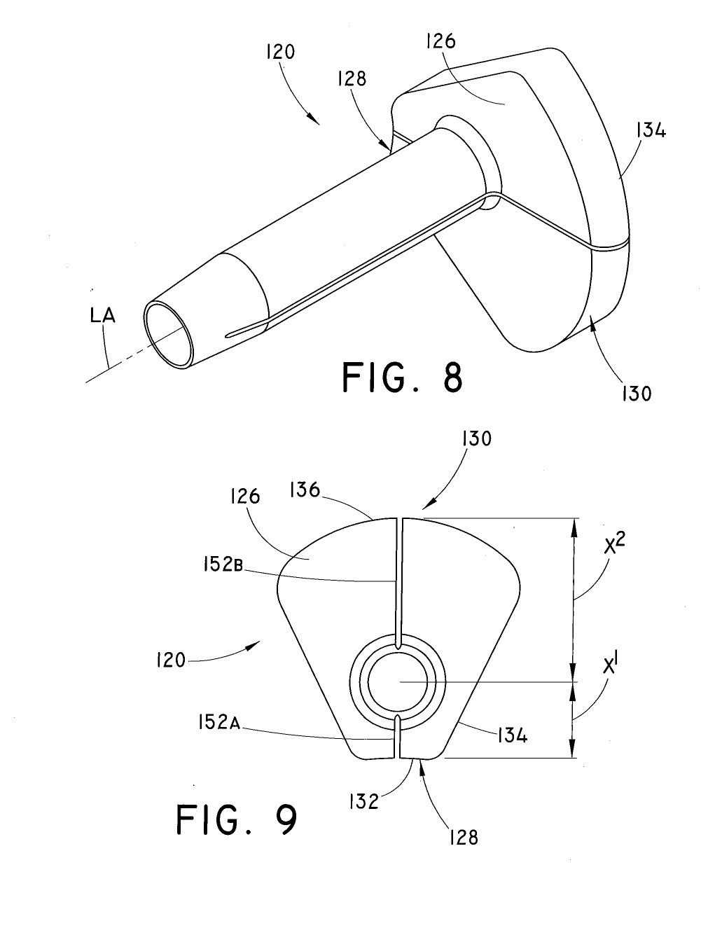

In FIGS. 2 and 4, the predetermined splittable region 50 includes a first splittable region 52 formed in the gripping member 26 and a second splittable region 54 formed in the elongated body 24. The first splittable region 52 can extend from the second end 38 of the elongated body 24 and outward to the periphery 56 of the gripping member 26. Although the periphery 56 is shown to have a circular shape, the periphery may have any other geometric shape such as elliptical, rectangular, triangular, or the like, or any irregular shape such as shown in FIGS. 8-9. The second splittable region 54 can extend from the second end 38 of the elongated body 24 proximate the gripping member 26 and at least partially along the elongated body 24 to the first end 36. The first splittable region 52 and the second splittable region 54 can be in communication with one another so that any separation along the first splittable region 52 can cause a separation along the second splittable region 54.

More than one splittable region may be formed in the gripping member 26 and/or the elongated body 24. For instance, FIG. 4 depicts a pair of first splittable regions 52 formed in the gripping member 26 at opposite sides of the opening 42, preferably about 180 degrees apart. The pair of first splittable regions can form a gripping member that is splittable into two removable portions 53A, 53B. Similarly, FIG. 5 depicts a pair of second splittable regions 54 may also be formed in the elongated body 24 at opposite sides of the inner chamber 34, preferably about 180 degrees apart. The pair of second splittable regions can form an elongated body that is splittable into two removable portions 55A, 55B. It is contemplated that three or more splittable regions may be formed into the gripping member and/or the elongated body, which are preferably spaced apart from one another equiangularly.

Each of the first and second splittable regions 52, 54 can be formed as a slot extending all the way through the wall, a score line, a groove, a series of perforations, or a reduced wall thickness region. It can be appreciated by those skilled in the art that the predetermined splittable region may include, besides generally longitudinal structures, other configurations such as helical, zipper-like or tongue-and-groove-like interface, or any other splittable connection interface along the contacting lateral edges.

In FIG. 4, one example of the first splittable region 52 can be defined as a longitudinal slot 60 that extends all the way through the wall thickness of the gripping member 26. The slot can be narrow relative to the size of the gripping member such that portions on both sides of the slot are in close proximity to one another. The slot 60 can extend from the periphery 56 and at least partially radially to the opening 42. An inner end 64 of the slot 60 may be terminated just short of the opening 42 so that a bridge 62 of material remains between the opening 42 and the end 64 of the slot. The inner end 64 of the slot 60 can be shaped to facilitate splitting along the elongated body, such as having a varied cross-section such as a V-shaped or U-shaped cross-section that leaves region adjacent the bridge 62 with an increasingly reduced wall thickness region.

In FIGS. 2 and 5, one example of the second splittable region 54 can be a longitudinal groove 70 formed in the outer wall surface 30, although a groove may be formed along the inner wall surface 32, in place of or in addition to the outer groove. The groove 70 can extend radially inward into the outer wall surface 30 such that a tubular ring 71 of material may be formed along the chamber 34. The ring 71 may be an extension of the bridge 62 so that the ring and the bridge are a continuous structure. The groove 70 may be extended longitudinally from the second end 38 of the elongated body 24, which is proximate the face of the gripping member 26, to at least partially to the first end 36. The groove 70 may be terminated short of the first end 36 so that a distal portion 72 of the elongated body 24 remains without a groove. The groove 70 can be shaped to facilitate splitting along the elongated body, such as having a varied cross-section such as a V-shaped or U-shaped cross-section that leaves the wall of the elongated body 24 with an increasingly reduced wall thickness region. An end 74 of the groove 70 corresponding with the second end 38 of the elongated body 24 can be in communication with the inner end 64 of the slot 60 to facilitate the separation of the retaining member into two or more removable portions. In particular the communication of the inner end of the slot and the end of the groove, can facilitate the formation of the two portions 55A, 55B of the elongated body and the two portions 53A, 53B of the gripping member.

The retaining member may include machined or molded components that are fused or bonded together, or may be integrally formed (i.e., to form a monolithic structure) by machining or molding process such as injection molding. The retaining member can be formed from any biocompatible material. The retaining member may be formed from a rigid and/or fracturable material, such as, e.g., polyamide (nylon) or acetal polyoxymethalyene (DELRIN™). The retaining member material can be configured to permit fracturing (e.g., snapping or breaking) along the predetermined splittable region 50 with application of a force. The material of the retaining member can be further configured to minimize breakage or flaking of the retaining member into multiple tiny pieces when fracturing, thereby reducing the possibility of such tiny pieces forming emboli in the body after procedure.

The retaining member may have a rigidity so that the gripping member maintains its shape and orientation relative to the elongated body during normal use. Such rigidity in the gripping member and the elongated body, can increase the manipulation of the retaining member when pushing the retaining member with the prosthesis within the vessel portion end opening. Further, a rigid retaining member can permit improved slidability of the retaining member over the prosthesis, as will be explained. When the retaining member is a unitized or monolithic rigid structure, the gripping member and the elongated body may also facilitate the interaction between the first and second splittable regions of the retaining member for removal thereof from the prosthesis at its target site. Furthermore, there is also a reduced risk of premature separation of any kind along the predetermined splittable region that may result in premature expansion of the prosthesis before being a desired target site. At least some of these features are particularly useful during an open surgical procedure to repair a damaged vessel because of the relatively small trauma pathway, which is also substantially transverse to the damaged vessel, that a clinician uses for repairing the damaged vessel.

Separation of the retaining member by fracturing with application of force at the gripping member can permit the elongated body to be removed from the prosthesis for rapid expansion thereof. The retaining member is less likely to cause movement of the prosthesis relative to the body vessel end portion, and can improve the anchoring location proximate the intended target site. It is contemplated that the fracturing of the elongated body can occur without peeling. When introducing the prosthesis into a body vessel end opening of a transected vessel, peeling may inadvertently move the respective end of the prosthesis longitudinally relative to the body vessel in closer proximity to the body vessel end opening. The prosthesis may also be moved radially away from the body vessel wall during peeling. This action may increase the risk of the prosthesis anchoring too close to the body vessel end opening, which can lead to an insufficient implantation and an increase of risk that the prosthesis may dislodge from the vessel after the procedure is completed. Further, dislodgement of the prosthesis during the peeling action may require reinsertion of the prosthesis, thereby prolonging the emergency procedure. Any additional time in a medical emergency or trauma procedure may prove to be fatal for the patient. Furthermore, when introducing the prosthesis into a body vessel end opening of a transected vessel, peeling may inadvertently distress healthy portions of the vessel wall and along the body vessel end opening when pulling apart the handles of the device. This may prolong the healing process, and could eventually cause necrosis of the distressed portion of the body vessel.

FIG. 6 depicts separation of an example rigid retaining member. The retaining member in phantom lines indicate the relative orientation of the components during the splitting process. Application of a force generally parallel to the longitudinal axis LA in a direction toward the first end 36 of the elongated body 24 (shown in phantom lines), represented by arrows 80, along a portion of the gripping member 26 (shown in phantom lines) will create a moment of inertia initially about the bridge and then along the split region. A force applied closer to the periphery 56 of the gripping member 26, rather than closer to the bridge, can create a greater moment of inertia about the bridge, which can make the separation easier. When the moment of inertia is sufficient to separate the portion of the retaining member 20 at the bridge initially into two bridge portions 62A, 62B, there can be a separation of the ring along the elongated body 24 into two ring portions 71A, 71B. As a result, the first and second retaining member portions 20A, 20B are formed and fully separated. Preferably, a moment of inertia sufficient to fracture the portion of the retaining member 20 at the bridge can cause fracturing of the ring along the elongated body 24 without peeling. Radial expansion forces of a self-expanding stent may facilitate the separation along the elongated body during the process of fracturing.

In one example, a retaining member comprises a polyamide material that is formed into a monolithic structure by injection molding. The overall length of the retaining member can be about 7-20 mm, with the thickness of the gripping member being about 2-5 mm and the length of the elongated body being about 5-15 mm. The outer diameter D1 of the elongated body 24 is about 3 mm and tapers to the inner diameter D2 of about 2 mm for the remaining 3 mm of the elongated body length. The periphery of the gripping member at its maximum extent that is measured from the longitudinal axis of the chamber has a distance of about 1.5 cm. In other words, when the gripping member has a circular shape, the overall diameter of the gripping member is about 3 cm. The prosthesis according to the aforementioned sizes can be used for about 6-8 mm diameter vessel. The length of the prosthesis can vary, which is dependent on the length of vessel removed between the transection vessel portions and the amount of insertion of the ends of the prosthesis within the vessel. In one example, the length of the prosthesis is about 40-50 mm. It is contemplated that at least some of the dimensions of the retaining member may be modified.

FIGS. 8-9 depict an alternative embodiment of a retaining member 120, which can include all of the aforementioned features described with respect to the retaining member 20. The shape of the gripping member 126 can be modified, such as a truncated wedge shape, to fit within tight spaces during delivery through the trauma pathway, while providing sufficient contact surface for the clinician to grasp. In one example, the gripping member 126 has a predefined distal end 128 to be inserted into the body first and a proximal end 130 to be handled by the clinician. A first segment 132 of the periphery 134 along the distal end 128 may have a lesser distance than a second segment 136 of the periphery 134 along the proximal end 130. Further, the distance X1 of the first segment 132 from the axis of the chamber can be less than the distance X2 of the second segment 136, and preferably substantially less than X2. The substantial size difference between the distance X1 and the distance X2 can be due to the amount of obstructions that may be present at the distal side of the body vessel opposite where the trauma pathway is formed by the clinician. A first 152A of the first splittable regions formed in the gripping member extends from the first segment 132 of the periphery 134, and a second 152A of the first splittable regions formed in the gripping member extends from the second segment 136 of the periphery 134. The general size of the second segment 136 and adjacent portions of the gripping member can be dependent on the amount of gripping area required to apply the force sufficient for separation.

FIGS. 10A-10H illustrate a method of vascular repair to a body vessel. FIG. 10A illustrates a body vessel 200, found for example in the leg of a patient, which has previously been subjected to a traumatic episode, resulting in a portion 202 of body vessel 200 being torn away or otherwise severely damaged. Pre-surgery preparation has been applied to the leg and a trauma pathway may be formed therein in order to gain external access to the body vessel and the damaged portion thereof. After clamping the body vessel 200 on both ends of the portion 202 to restrict blood flow temporarily, the body vessel 200 can be cut or transected by the clinician into two portions 200A, 200B, as shown in FIG. 10B. The transection may be at the damaged portion 202 of the blood vessel or as far away as necessary from the damaged portion to remove unhealthy portions of the body vessel or unrepairable portions of the body vessel. Sutures 204 can be attached to the end openings 205 of body vessel portions 200A, 200B to keep them secured in a relatively fixed position and opened to facilitate insertion of the introducer with the prosthesis. Forceps may also be used in a similar manner. Any number of sutures can be used to retain the end openings 205 in the open position, although triangulation sutures can be sufficient, with each suture being about 120 degrees apart from the adjacent suture. A prosthesis 12 is selected to have a radial expanded cross-section and a longitudinal length sufficient to bridge the body vessel portions 200A, 200B and radially fit within the body vessel portions.

According to FIGS. 10C-10D, the introducer system 10 having the first retaining member 20 coupled to the first end 14 of the prosthesis 12, and the second retaining member 22 (shown in FIG. 10F) coupled to the second end of the prosthesis, can be situated and oriented adjacent the body vessel portion 200A through the trauma pathway. The elongated body 24 of the first retaining member 20 with the first end 14 of the prosthesis 12 can be inserted into vessel portion 200A by a sufficient distance for anchoring into the body vessel portion. When the retaining member is a rigid member, pushability of the retaining member at the gripping member can be easier than when the gripping member of the retaining member is made of a more flexible material. Preferably, when the retaining member is a rigid member, there is a reduced risk of premature separation of any kind along the predetermined splittable region during manipulation of the retainer member that may result in premature expansion of the prosthesis before being a desired target site. It is preferred that the vessel portion initially selected be the non-blood supplying vessel end. Vessel portion 200A may be manually pulled over the elongated body 24. The first retaining member 20 can be oriented within the vessel portion such that the first splittable region 52 extends in the direction of the trauma pathway. In this fashion, the clinician can grasp the proximal portions of the gripping member on the both sides of the first splittable region to initiate separation.

In FIG. 10E, after insertion of the elongated body 24 into the end opening 205 of the first portion 200A of transected body vessel 200, the first retaining member 20 can be removed from the first end 14 of the prosthesis 12. This can permit expansion and purchase of a portion of the prosthesis 12 along the wall of the vessel portion 200A. The retaining member 20 can be separated into two or more removable portions as described herein when within the vessel with preferably minimal to no movement of the retaining member away from the vessel end opening. Separation can occur, e.g., by snapping or breaking the elongated body 24 and the gripping member 26 to fracture along the pre-determined splittable region 50 with application of force at the gripping member 26. As a result, the elongated body 24 is permitted to be rapidly removed from the first end 14 of the prosthesis 12 without peeling, thereby allowing for rapid expansion of the prosthesis to anchor into the vessel wall, and resulting into two portions 20A, 20B of the retaining member that can be easily removed from the body. The first and second retaining member portions 20A, 20B can be removed from the body vessel after expansion by withdrawing the retaining member portions at the gripping portions, and pulling the portions out from the body vessel.

According to FIG. 10F, the vessel portion 200A is now sealably engaged to the first end 14 of the prosthesis 12. The introducer system 10 can then be manipulated in order to introduce the elongated body of the second retaining member 22 with the second end of the prosthesis 12 into vessel portion 200B by a sufficient distance for the purposes of anchoring. After insertion, the second retaining member 22 can be removed from the second end 16 of the prosthesis 12 such as shown for the first retaining member 20 in FIG. 10E. This can permit expansion and purchase of a portion of the prosthesis 12 along the wall of the vessel portion 200B.

In FIG. 10G, vessel portion 200B is now be sealably engaged to the second end 16 of the prosthesis 12. Accordingly, the prosthesis 12 is fully deployed to bridge first and second portions 200A, 200B of the transected body vessel 200 to form a conduit for blood flow. Sutures 204 can then be removed. Preferably, portions of the exterior surfaces of the prosthesis sealably engage with the luminal walls of the body vessel to inhibit leakage of blood and to force blood to flow throughout the body vessel during emergency surgery, and particularly to obtain hemostasis while maintaining blood perfusion. FIG. 10H shows the prosthesis 12 deployed and connecting body vessel portions 200A, 200B within the leg of the patient. The prosthesis 12 can be adapted for permanent placement within the patient, thereby obviating a need for subsequent surgical intervention

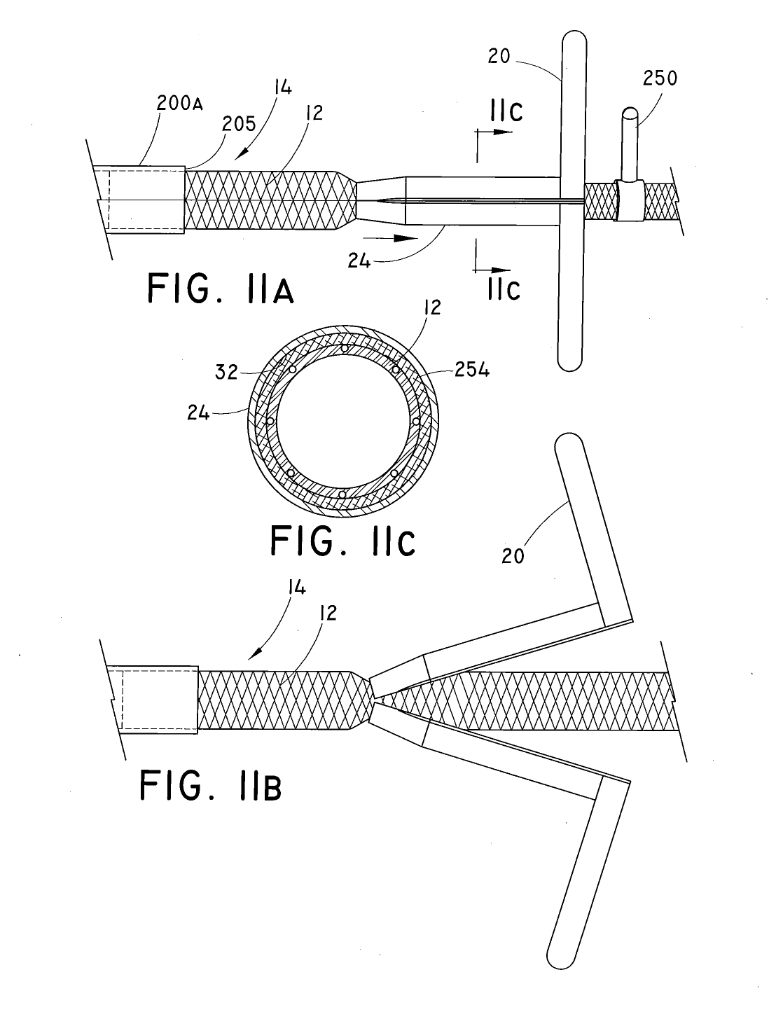

Removal of the retaining members from the respective ends of the prosthesis is preferably performed by fracturing the retaining member, as described herein, while the retaining member is within the vessel portion as shown in FIG. 10E. Alternatively, as shown in. FIG. 11A, the retaining member may be fractured after sliding the retaining member from the respective end away from the vessel portion end opening. To remove by sliding, a clamping mechanism 250 or even the fingers of the clinician may compress and/or hold the prosthesis in place relative to the moving retaining member. The retaining member can be at least partially, and preferably completely, removed from the vessel portion end opening 205. When the retaining member is a rigid member, slidability of the retaining member using the gripping member over the prosthesis can be easier than when the gripping member of the retaining member is made of a more flexible material. As a result, the first end of the prosthesis can begin to expand against the vessel wall. FIG. 11B depicts the separation of the retaining member as described herein. To facilitate removal and/or slidability, a lubricious liner or coating 254 may be applied along the inner wall surface 32 of the elongated body 24 and/or the edge that defines the opening 42, as shown in FIG. 11C. The lubricious liner or coating can include a fluoropolymer, such as polytetrafluoroethylene (PTFE) or fluorinated ethylene propylene (FEP). The lubricious material can provide a slippery, low friction surface to enhance slidability of the inner surface of the retaining member along the prosthesis. The outer surface of the retaining member, such as the elongated body can be coated with the lubricious liner or coating to facilitate removal of the retaining member from the body vessel.

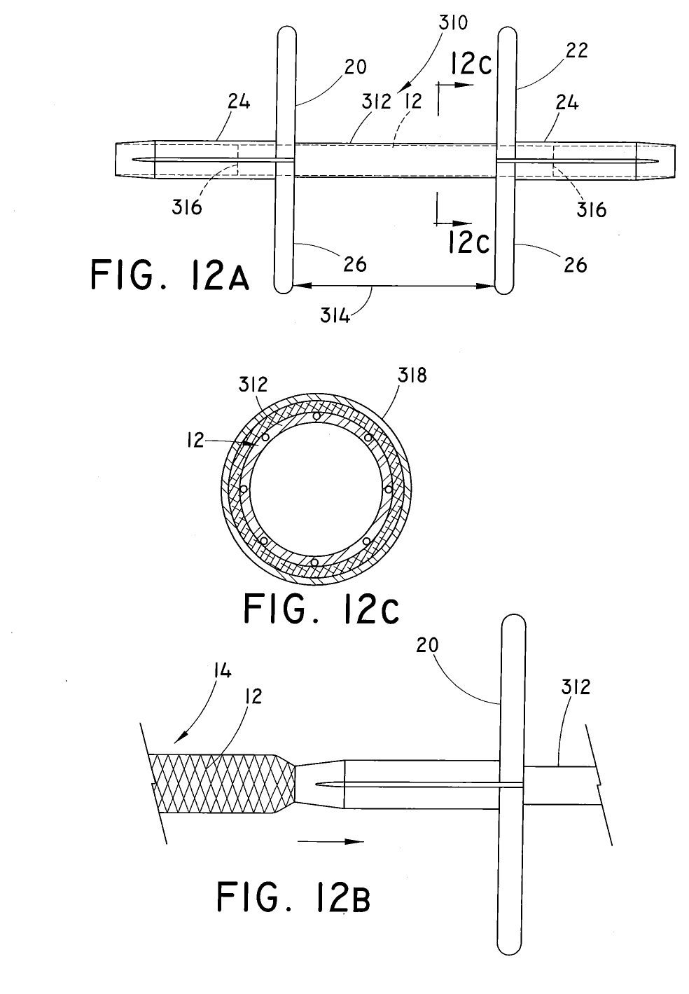

FIGS. 12A-12C illustrate another embodiment of the introducer system 310. The system 310 includes the prosthesis 12 and the first and second retaining elements 20, 22. In addition, the system 310 includes a retaining sleeve 312 surrounding at least an intermediate portion of the prosthesis 12. The sleeve 312 is configured to retain the corresponding portion of the prosthesis in a radially compressed configuration. The sleeve can include a thin layer of a polymeric material, such as a polyether block amide (PEBA), polyamide (nylon), polyurethane, PTFE, and the like. The length of the sleeve 312 is sufficiently long to allow the retaining member to slide thereover. The sleeve length may extend entirely across the intermediate section 314 of the prosthesis 12 that is between the retaining members 20, 22. In another example, one sleeve per retaining member can be used. Here, each sleeve can have a length for the retaining member to slide along the sleeve, yet a segment of the prosthesis between adjacent ends of the sleeves can remain uncovered.

The ends 316 of sleeve 312 may extend at least partially within the elongated body 24, as show by the dashed lines in FIG. 12A, terminating at some point between the first and second ends of the elongated body. Preferably, the sleeve extends to a point closer to the second end than the first end of the elongated body so that when the retaining member is removed the end of the prosthesis can immediately expand to anchor into the vessel wall, such as shown in FIG. 12B. The sleeve 312 can retain the prosthesis to a profile that is sized to permit the retaining member to slide more easily and/or for better manipulation of the introducer system.

As shown in FIG. 12C, the outer surface of the sleeve 312 and/or the inner surface of the elongated body may include a lubricious liner or coating 318 (shown by phantom lines) to facilitate slidability, such as described above. The sleeve 312 may then be removed from the prosthesis, which preferably occurs after removal of the retaining members. In one example, the sleeve 312 is a splittable sleeve. Examples of splittable sheath configurations can be found in U.S. Pat. No. 6,447,540 to Fontaine et al. and U.S. Pat. No. 6,827,731, each of which is incorporated herein by reference in its entirety. In one example, the sheath can comprise a splittable polymer such as molecularly oriented, non-isotropic PTFE that is used to make the PEEL-AWAY® Introducer Sheath (Cook Incorporated, Bloomington, Ind.), which is described in, e.g., U.S. Pat. No. 4,306,562 to Osborne and U.S. Pat. No. 4,581,025 to Timmermans, each of which is incorporated herein by reference in its entirety. FIG. 12B depicts the retaining member 20 sliding along the sleeve 312. Similar to FIG. 11A, a clamping mechanism or even the fingers of the clinician may need to hold the prosthesis in place relative to the moving retaining member.

It is further contemplated that when the prosthesis is a mechanically expandable structure, an expandable device, such as a balloon expandable device, can be inserted into the prosthesis after removal of the retaining member. The expandable device can be expanded within the prosthesis to expand the prosthesis against the body vessel, as can be appreciated by those skilled in the art.

Drawings in the figures illustrating various embodiments are not necessarily to scale. Some drawings may have certain details magnified for emphasis, and any different numbers or proportions of parts should not be read as limiting, unless so-designated in the present disclosure. Those skilled in the art will appreciate that embodiments not expressly illustrated herein may be practiced within the scope of the present invention, including those features described herein for different embodiments may be combined with each other and/or with currently-known or future-developed technologies while remaining within the scope of the claims presented here. It is therefore intended that the foregoing detailed description be regarded as illustrative rather than limiting. And, it should be understood that the following claims, including all equivalents, are intended to define the spirit and scope of this invention.

1. An introducer system for a prosthesis for repair of two vessel portions of a transected body vessel, comprising:
a prosthesis having a first end and a second end, the prosthesis being movable between a radially compressed configuration and a radially expanded configuration; and
a retaining member fitted over at least one of the first and second ends of the prosthesis to retain a corresponding length of the prosthesis in the radially compressed configuration, the retaining member comprising an elongated body for insertion into a vessel portion of a transected body vessel, the elongated body having a chamber formed therein to receive the respective end of the prosthesis, a gripping member extending outward from the elongated body, and a splittable region formed in the elongated body and the gripping member, wherein application of a force to the gripping member separates the gripping member and the elongated body along the splittable region to permit movement of the corresponding length of the prosthesis to the radially expanded configuration for engagement with the vessel portion.

2. The system of claim 1, wherein the splittable region comprises a first predetermined splittable region formed in the gripping member, and a second predetermined splittable region formed in the elongated body.

3. The system of claim 2, wherein the elongated body further comprises a first end configured to be initially inserted into the vessel portion, and a second end, the chamber extending at least between the first end and the second end of the elongated body, wherein at least one second predetermined splittable region is formed along a surface of the elongated body.

4. The system of claim 3, wherein the at least one first predetermined splittable region further comprises a slot extending through a thickness of the gripping member.

5. The system of claim 3, wherein the at least one second predetermined splittable region further comprises a groove formed in an outer surface of the elongated body.

6. The system of claim 3, wherein the at least one first predetermined splittable region comprises a pair of first predetermined splittable regions, wherein one of the first predetermined splittable regions has a longer length than the other of the first predetermined splittable regions.

7. The system of claim 1, wherein the elongated body includes an inner surface having a lubricious layer.

8. The system of claim 1, wherein the retaining member has a rigidity suitable to maintain a relative orientation between the gripping member and the elongated body.

9. The system of claim 1, wherein the elongated body and the gripping member is configured to fracture along the splittable region upon application of a force to the gripping member.

10. The system of claim 2, wherein the gripping member further comprises a periphery, and an opening formed in the gripping member and in communication with the chamber, wherein at least one first predetermined splittable region is formed along a surface of the gripping member, the at least one first predetermined splittable region extending from the periphery and terminating at a point closer to the opening than the periphery, wherein the at least one first predetermined splittable region is configured to place portions on both sides of the at least one first predetermined splittable region in close proximity to one another.

11. The system of claim 1, further comprising a sleeve at least partially surrounding an intermediate portion of the prosthesis that extends beyond the retaining member, the sleeve configured to retain the intermediate portion in the radially compressed configuration.

12. An introducer system for a prosthesis to connect two vessel portions of a transected body vessel, comprising:
a prosthesis having a first end and a second end, the prosthesis being movable between a radially compressed configuration and a radially expanded configuration; and
a first retaining member and a second retaining member fitted over the first and second ends of the prosthesis, respectively, to retain corresponding lengths of the prosthesis in the radially compressed configuration,
each of the first and second retaining members comprising a gripping member and an elongated body extending longitudinally from the gripping member, the gripping member having an opening formed therein and at least one first splittable region formed therein, the elongated body having a tubular chamber formed therein in communication with the opening of the gripping member to receive the respective end of the prosthesis, and at least one second splittable region formed therein, wherein the at least one second splittable region is in communication with the at least one first splittable region,
wherein the retaining member has a rigidity suitable to maintain a relative orientation between the gripping member and the elongated body, wherein application of a force to the gripping member separates the gripping member and the elongated body along the splittable region to permit movement of the corresponding length of the prosthesis to the radially expanded configuration for engagement with the vessel portion.

13. The system of claim 12, further comprising a removable sleeve at least partially surrounding an intermediate portion of the prosthesis that is between the first and second retaining members, the sleeve configured to selectively retain the intermediate portion of the prosthesis in the radially compressed configuration.

14. The system of claim 13, wherein the sleeve extends from the intermediate portion of the prosthesis to at least partially within the elongated body of at least one of the first and second retaining members.

15. The system of claim 14, wherein the sleeve includes an outer surface having a lubricious layer.

16. A method of connecting two vessel portions of a transected body vessel, comprising:
introducing a first end of a prosthesis in a radially compressed configuration, retained by a tubular body of a retaining member, into an end opening of one of the two vessel portions, the retaining member having a gripping member extending outwardly from the elongated body, and a splittable region formed in the elongated body and the gripping member; and
applying a force to the gripping member to separate the retaining member along the splittable region into two or more removable portions, whereby the first end of the prosthesis is permitted to move to a radially expanded configuration for engagement with the vessel portion.

17. The method of claim 16, further comprising the step of introducing a second end of a prosthesis in a radially compressed configuration, retained by a tubular body of a second retaining member, into an end opening of the other of the two vessel portions, the second retaining member having a gripping member extending outwardly from the elongated body, and a splittable region formed in the elongated body and the gripping member; and
applying a force to the gripping member to separate the second retaining member along the splittable region into two or more removable portions, whereby the second end of the prosthesis is permitted to move to a radially expanded configuration for engagement with the vessel portion.

18. The method of claim 17, further comprising the step of removing the removable portions of the retaining member and the second retaining member from the respective vessel portions by sliding each removable portion over the prosthesis and out of the vessel portion.

19. The method of claim 17, wherein each of the retaining member and the second retaining member has a rigidity suitable to maintain a relative orientation between the gripping member and the elongated body.

20. The method of claim 17, further comprising the steps of:
providing a removable sleeve at least partially surrounding an intermediate portion of the prosthesis, and extending at least partially within at least one of the retaining member and the second retaining member, the sleeve configured to selectively retain the intermediate portion in the radially compressed configuration; and
sliding the retaining member over the prosthesis and over at least a portion of the removable sleeve and out away from the vessel portion prior to the applying a force step.
